(19) United States
(12) Patent Application Publication
(10) Pub. No.: US 2010/0140372 A1
(43) Pub. Date: Jun. 10, 2010

(54) Toilet paper roll scented foam insert

(76) Inventor: David O. Patrick, Jackson, MO (US)

Correspondence Address: LITMAN LAW OFFICES, LTD., POST OFFICE BOX 41200, SOUTH STATION, ARLINGTON, VA, 22204, US

(21) Appl. No.: 12/591,942

(22) Filed: Dec. 4, 2009

Related U.S. Application Data
(60) Provisional application No. 61/193,600, filed on Dec. 9, 2008.

(51) Int. Cl. A61L 9/04

(52) U.S. Cl. 239/52

(57) ABSTRACT

The toilet paper roll scented foam insert is a pliable, scent-impregnated foam sleeve that slips over the roller of a toilet paper dispenser and resiliently grips the roller. The insert is dimensioned and configured to slide through the hollow core of a roll of toilet paper. The sleeve has dentate ends that extend beyond the ends of the roll of toilet paper. When the roller is rotated to dispense paper, the passage of air through the dentate ends helps to dispense and distribute the scent through the air adjacent the toilet to overcome toilet odor.

### CROSS-REFERENCE TO RELATED APPLICATION

This application claims the benefit of U.S. Provisional Patent Application Ser. No. 61/193,600, filed Dec. 9, 2008.

### BACKGROUND OF THE INVENTION

1. FIELD OF THE INVENTION

The present invention relates generally to toilet paper dispensing accessories. More specifically, the invention relates to a toilet paper roll scented foam insert that provides a scent during rollout and use of the toilet paper.

2. DESCRIPTION OF THE RELATED ART

Every modern bathroom is equipped with a toilet paper dispenser for dispensing disposable toilet tissue from a roll for hygienic purposes. Nevertheless, using the bathroom may still prove unpleasant because of the odors associated with the toilet. Many bathrooms are equipped with an exhaust fan to help alleviate this problem. However, such exhaust fans provide only a partial solution to the problem. Stand-alone air fresheners are of some help. But stand-alone air fresheners often go stale, aren't replaced regularly, lack sufficient strength for overcoming toilet odors, and are frequently knocked over, even if there's a place to put them in the bathroom. There is a need for a better solution to the problem of toilet odor in the bathroom.

Thus, a toilet paper roll scented foam insert solving the aforementioned problems is desired.

### SUMMARY OF THE INVENTION

The toilet paper roll scented foam insert is a pliable, scent-impregnated foam sleeve that slips over the roller of a toilet paper dispenser and resiliently grips the roller. The insert is dimensioned and configured to slide through the hollow core of a roll of toilet paper. The sleeve has dentate ends that extend beyond the ends of the roll of toilet paper. When the roller is rotated to dispense paper, the passage of air through the dentate ends helps to dispense and distribute the scent through the air adjacent the toilet to overcome toilet odor.

These and other features of the present invention will become readily apparent upon further review of the following specification and drawings.

### BRIEF DESCRIPTION OF THE DRAWINGS

FIG. 1 is an environmental, perspective view of a toilet paper roll scented foam insert according to the present invention.

FIG. 2 is a perspective view of the scented foam insert according to the present invention.

FIG. 3 is a front view of the scented foam insert according to the present invention.

FIG. 4 is a plan view of the scented foam insert, unrolled, according to the present invention.

Similar reference characters denote corresponding features consistently throughout the attached drawings.

### DETAILED DESCRIPTION OF THE PREFERRED EMBODIMENTS

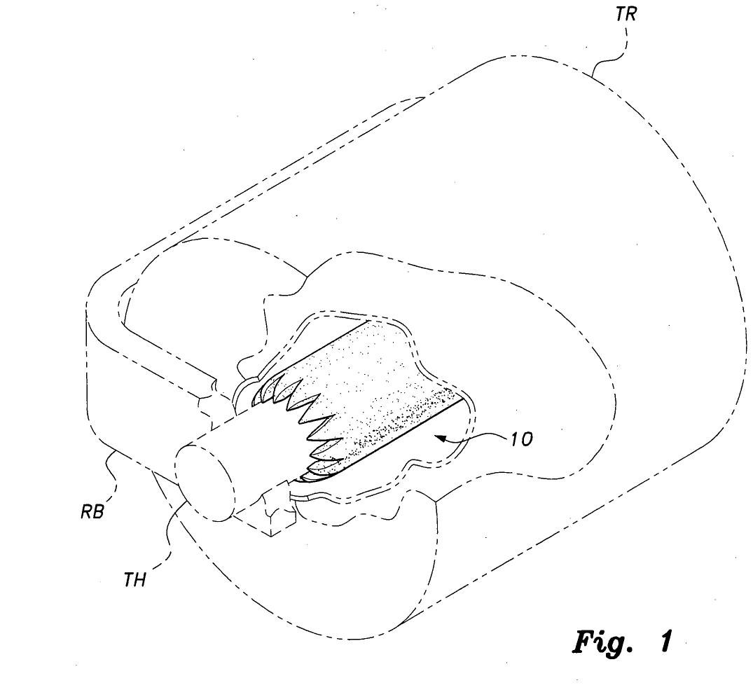

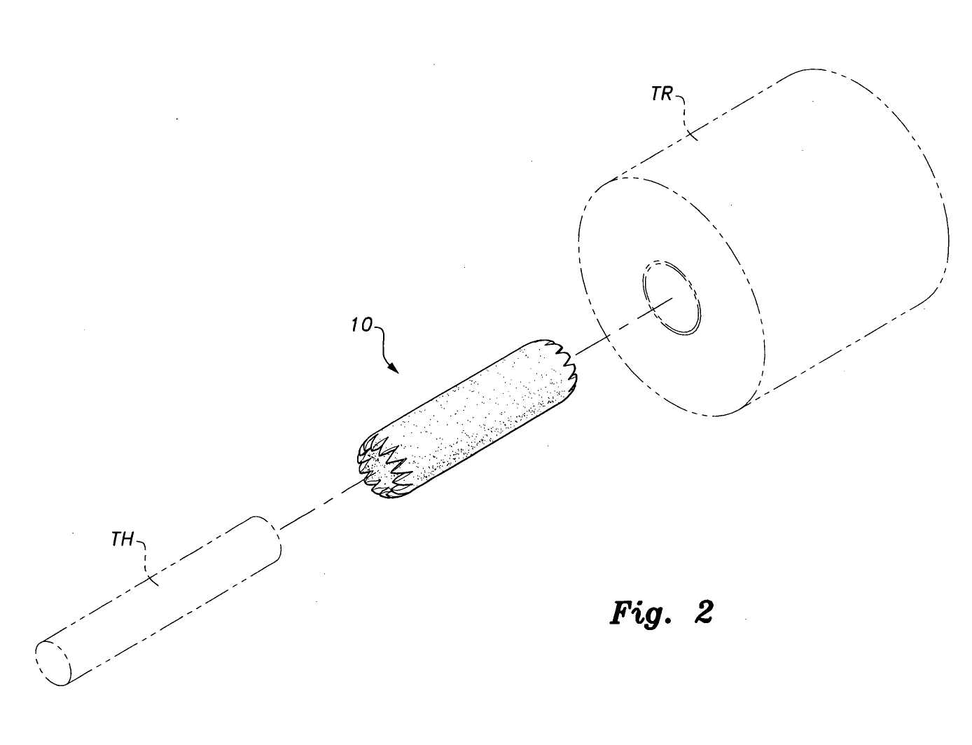

As shown in FIGS. 1-3, the toilet paper roll scented foam insert, designated generally as 10 in the drawings, is a pliable, scent-impregnated, hollow, foam, substantially cylindrical sleeve that can be placed on the roller TH of a toilet paper dispenser RB. The sleeve is preferably sufficiently resilient that it grips the roller. The roller TH can then be inserted through the hollow core of a roll TR of toilet paper and mounted on the dispenser RB. The cylindrical insert 10 has dentate ends that define teeth or vanes 15.

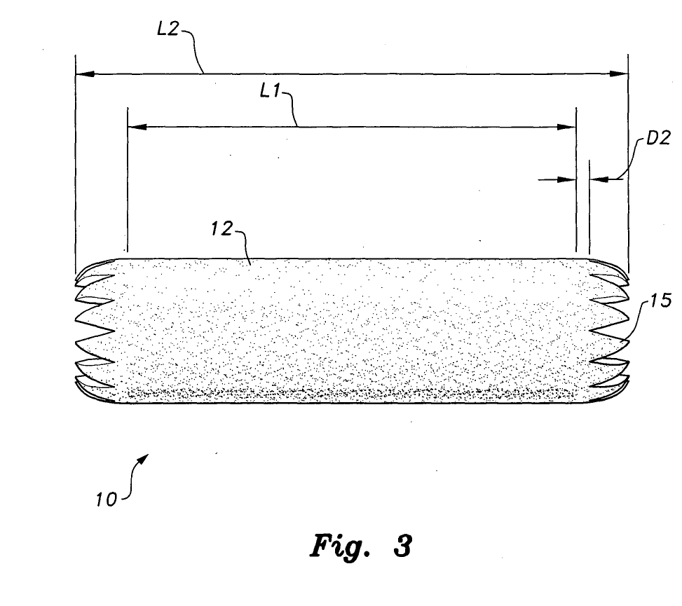

As most clearly shown in FIGS. 1 and 3, the dimensions of L1 , L2, and D2 are formulated so that the vanes 15 extend beyond the ends of a toilet paper roll TR, preferably by about ⅜″ on each side. During rollout of the paper, the rotating vanes 15 facilitate dispersal of the scent, the teeth or vanes 15 maximizing the surface area exposed to the air during rotation of the roller. The foam sleeve 10 emanates a pleasant fragrance during rollout and use of the paper in order to mask or neutralize toilet odors.

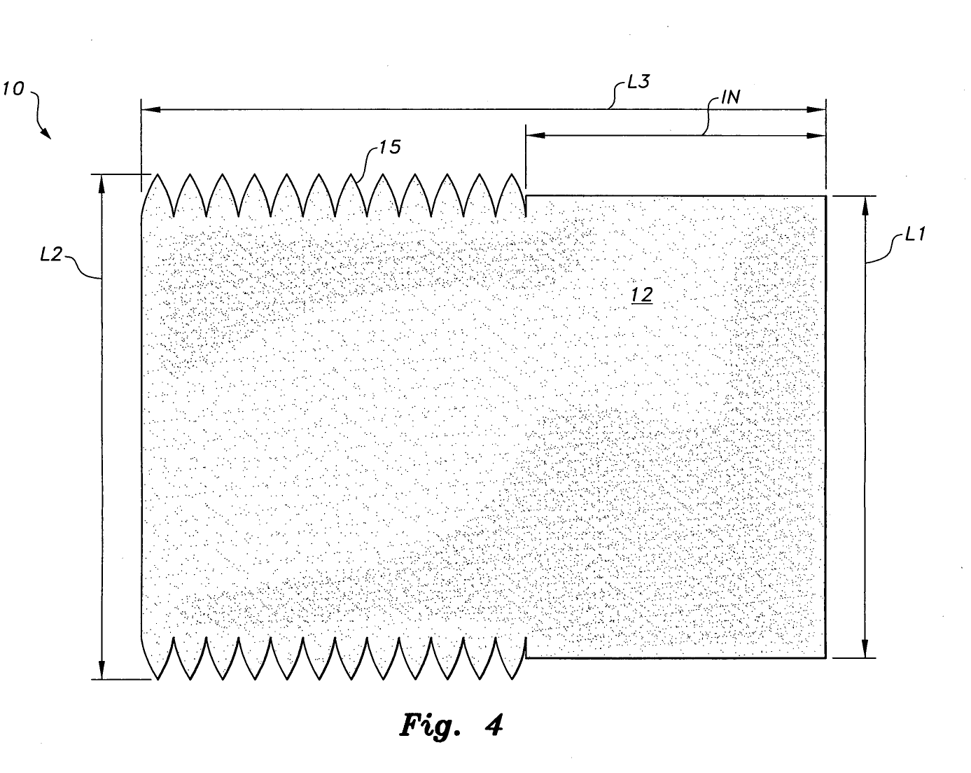

As shown in FIG. 4, the preformed shape 12 of insert 10 is substantially planar and rectangular having a total length L3 and a total width L2. Preferable total length L3 is approximately 8 inches. Preferable total width L2 is approximately 5.9062 inches. Preferable width L1 of portion IN is approximately 5.4063 inches. Portion IN preferably extends 3.5000 inches beyond dentate portion 15. During manufacturing, when the device 10 is formed into a roll, extended portion IN is tucked inside the roll and glued or otherwise fastened to remainder of inside roll portion to stably affix the roll into a cylindrical form.

The insert 10 may be made from any suitable foam material, such as foam rubber, polyurethane, polystyrene, or other resilient foam material. The scent may be any suitable fragrance, natural or synthetic.

It is to be understood that the present invention is not limited to the embodiment described above, but encompasses any and all embodiments within the scope of the following claims.

I claim:

1. A toilet paper roll scented foam insert, comprising:
a cylindrical, hollow sleeve made from a resilient foam material, the sleeve having opposing dentate ends defining vanes; and
a fragrance, the sleeve being impregnated with the fragrance;
wherein the sleeve is dimensioned and configured for being placed on a roller of a toilet paper dispenser, the vanes extending beyond opposite ends of a toilet paper roll mounted on the roller, whereby dispersal of the fragrance from the insert into ambient air is accelerated when the roller is rotated in order to mask toilet odors.

2. The toilet paper roll scented foam insert according to claim 1, wherein the foam insert is formed from a substantially planar and rectangular piece having a length extending into a tail portion, the tail portion being rolled and tucked inside the foam insert to stably affix the roll into a cylindrical form.

3. The toilet paper roll scented foam insert according to claim 2, wherein the total length of the planar rectangular piece is approximately eight inches.

4. The toilet paper roll scented foam insert according to claim 2, wherein the total width of the planar rectangular piece is approximately 5.9062 inches.

5. The toilet paper roll scented foam insert according to claim 2, wherein the length of the tail portion is approximately 3.5000 inches.

6. The toilet paper roll scented foam insert according to claim 2, wherein the tail portion has a width of approximately 5.4063 inches.
